(19) United States
(12) Patent Application Publication
(10) Pub. No.: US 2023/0024050 A1
(43) Pub. Date: Jan. 26, 2023

(54) Dermatological Compositions and Methods of Treatment against Cutaneous T-Cell Lymphoma

(71) Applicant: Soligenix, Inc., Princeton, NJ (US)

(72) Inventors: Oreola Donini, Coquitlam (CA); Richard Straube, Princeton, NJ (US); Christopher Schaber, Princeton, NJ (US)

(21) Appl. No.: 17/860,599

(22) Filed: Jul. 8, 2022

Related U.S. Application Data
(60) Provisional application No. 63/219,765, filed on Jul. 8, 2021.

(51) Int. Cl. A61K 31/122; A61K 41/00; A61N 5/06

(52) U.S. Cl. CPC A61K 31/122; A61K 41/0057; A61N 5/062

(57) ABSTRACT

A method of treating CTCL comprising applying a combination of an effective amount of hypericin together with a form of visible light photodynamic therapy. Preferably, the effective amount of hypericin is an ointment comprising less than 1% hypericin. More preferably, the form of photodynamic therapy comprises an administration of escalating doses of visible light. Optionally, the escalating doses of visible light starts at about 5 J/cm² and increases to a maximum dose of about 12 J/cm².

### RELATED APPLICATIONS

This application is a U.S. Patent Application claiming priority from U.S. Provisional Application No. 63/219,765 filed Jul. 8, 2021.

### FIELD OF THE INVENTION

The invention relates to the field of dermatological compositions and methods of administration of same for the treatment of cutaneous T-cell lymphoma.

### BACKGROUND OF THE INVENTION

Hypericin is a known photodynamic agent with the potential to treat a variety of inflammatory dermatological diseases associated with lymphocytic infiltrates, including cutaneous T-cell lymphoma (CTCL). In the presence of light irradiation, hypericin excites oxygen to its singlet state and is capable of generating superoxide radicals that can lead to oxidation of tryptophan imidazole groups in proteins and to oxidation of fatty acids in biological systems, and ultimately to cellular apoptosis. Hypericin is maximally activated by light of about 500-650 nM wavelength, i.e., in the yellow-red region of the electromagnetic spectrum (Meruelo et al, 1988; Thomas et al, 1992; Lavie et al, 1989; Head et al, 2006).

Importantly, hypericin is preferentially taken up by malignant cells compared to healthy cells and even more so by malignant T-cells, enabling significant selectivity in its use in CTCL, in addition to its localized application (Fox et al, 1998; Xu et al, 2019).

The mechanism by which topical photoactivated hypericin provides clinical benefit to treat inflammatory skin diseases, characterized by lymphocytic infiltrates, includes generation of singlet oxygen and apoptosis. This is a similar mechanism to that of other agents, such as 5-aminolevulinic acid and other porphyrins used in photodynamic therapy that are known to have clinical efficacy in the treatment of inflammatory skin diseases. Typically, apoptosis mediated by reactive oxygen species is executed predominantly via the intrinsic (mitochondrial) pathway. This is particularly advantageous for disorders such as CTCL that have been shown often to exhibit defective extrinsic (death receptor mediated) apoptotic pathways including FAS and TRAIL. Also, studies on mechanism of antitumor activity indicate that hypericin binds to the chaperone protein, heat shock protein 90, leading to its ubiquitinylation (Barliya et al, 2011). This disrupts several critical client proteins that regulate cell growth pathways resulting in their de-stabilization, rapid degradation, and elimination ultimately leading to cell death. Additionally, hypericin is known to be a potent protein kinase C inhibitor, thereby disrupting cell signaling mechanisms and inducing apoptosis (Barliya et al, 2011; Chan et al, 2009; Dzurova et al, 2014; Kocanova et al, 2006; Takahashi et al, 1989; Zhang et al, 1997).

Treatment of CTCL in patients is primarily determined by disease extent and the impact on quality of life. Early-stage disease, with disease primarily confined to the skin, has a favorable prognosis, with skin-directed therapies most often used as first-line treatment. Skin-directed therapies for treating the early stages of disease (IA to IIA) include topical corticosteroids, topical mechlorethamine, topical bexarotene, and ultraviolet (UV) phototherapy. Total skin electron beam therapy (TSEBT) and localized superficial radiotherapy are also considered skin-directed therapies. Prolonged complete remissions have been obtained, although disease cure is unclear.

Advanced stage disease (MF stages IIB-IVB, SS) is often treatment refractory and results in an unfavorable prognosis; treatment is usually systemic and is aimed at reducing the tumor burden, delaying disease progression, and preserving quality of life. Current approaches include immune-biologic and targeted therapies, but the duration of clinical response is often short and survival benefits observed to date have been modest.

Therapies specifically approved for treatment of CTCL are primarily for patients with advanced/late-stage disease (˜5% CTCL patients). The most recently EMA approved product, Poteligeo® (mogamulizumab), is indicated after other systemic therapies have failed. This product comes with significant warnings and precautions for use including dermatological reactions, infusion reactions, complications of HSCT, tumor lysis syndrome, large cell transformation, cardiac disorders, diarrhea, upper respiratory tract infections and thrombocytopenia. Roferon A© (Interferon alfa-2A) may be used for treatment of patients with stage IIB and above. This product also comes with significant warnings including psychiatric disorders, infections, bone marrow suppression as well as endocrine, hepatic and autoimmune effects.

The PROspective Cutaneous Lymphoma International Prognostic Index (PROCLIPI) database primarily investigates disease course and prognostic factors in MF. However, a recently published analysis from this international registry focused on identifying (i) differences in first-line approaches according to staging; (ii) parameters related to a first-line systemic approach and (iii) response rates and quality of life measures (Quaglino et al, 2021).

In total 395 patients were included (50% stage IA, 42% stage IB, 8% stage IIA) in this analysis, recruited from 41 centers across 17 countries. European centers accounted for 88% of the patients. The most common form of first-line therapy was found to be skin-directed therapy (322 cases, 81.5%), while a smaller percentage (44 cases, 11.1%) received systemic therapy. Analysis demonstrated that systemic therapy was associated with use in patients with a higher clinical stage, presence of plaques, higher modified Severity Weighted Assessment Tool (mSWAT) and folliculotropic ME.

It was found that the overall response rate (ORR) to first-line skin-directed therapy was significantly better compared to systemic therapies (73% versus 57%; P=0.027). HRQoL was found to improve significantly both in patients with responsive disease and in those with stable disease, highlighting the importance of incorporating quality of life measurements into assessments of treatment activity. It was concluded that future treatment guidelines should address high-risk disease characteristics such as presence of plaques and folliculotropic MF, along with quality of life evaluation, as these are important features that drive treatment decisions (Quaglino et al, 2021).

The following skin-directed therapies are currently known in the art:

Steroids: Corticosteroids are frequently used in early disease and as adjunctive therapy in more advanced stages of the disease. Their multiple effects include induction of apoptosis, impact on lymphocyte adhesion to endothelium, and the downregulation of transcription factors (nuclear factor-kB and activator protein-1) with decreased cytokine, adhesion molecule, and growth factor production. Topical steroids also decrease erythema, scaling, and pruritus in erythrodermic CTCL. High potency steroids are usually required, precluding use for more than 1-2 months. Side effects associated with long-term use include skin atrophy, hypopigmentation, striae, and potential systemic absorption with adrenal suppression.

Mechlorethamine: Topical nitrogen mustard (mechlorethamine hydrochloride) (NM) is an alkylating agent. Topical NM applications are commonly used for early-stage disease and work by inducing DNA damage. Ledaga® is approved for the topical treatment of mycosis fungoides-type cutaneous T-cell lymphoma (MF-type CTCL) in adult patients. However, only 11% of patients maintained a Complete Response (CR) after 1 year. Skin clearance often requires greater than 6 months of treatment and is usually followed by maintenance therapy, although there is no evidence that prolonged maintenance reduces recurrence. Cutaneous side effects, including burning, pruritus, and irritant or allergic contact dermatitis, are common and often result in treatment cessation. There is a small increased risk (1-5%) of developing non-melanoma skin cancers (NMSCs). Patients must also ensure no contact of treated skin with family members, leading to significant intimacy issues.

Bexarotene: Bexarotene is a synthetic retinoid with the oral form (i.e., capsules) selectively binding retinoid X receptor (RXR) isoforms, affecting cell differentiation and inducing apoptosis. A topical bexarotene 1% gel is approved in the USA but not in the EU, for the treatment of early-stage CTCL (up to 4 times daily) which likely also induces local cell apoptosis. Topical bexarotene is recommended twice daily; high rates of irritation are seen. Responses were seen in many patients (stage IA-IIA) after a median of 20 weeks of treatment (ORR, 63%; CR, 21%). Long-term treatment is limited by irritation. Overall use in CTCL is limited due to the marginal efficacy coupled with accessibility issues.

Photodynamic Therapy: Psoralen plus ultraviolet light A (PUVA) has an established benefit in early-stage CTCL and involves oral 8-methoxy psoralen (8-MOP), which sensitizes the skin to ultraviolet A (UVA) light radiation (320-400 nm), inducing tumor cell apoptosis and DNA damage, suppressing keratinocyte cytokine production, and depleting Langerhans cells. PUVA is approved for psoriasis, but not for CTCL. The initial UVA light dosage is approximately 0.5 J/cm2, increasing as tolerated, and given 3 times weekly until CR is achieved. Proper eye protection is needed for 12 to 24 hours after treatment sessions for cataract prevention. Maintenance therapy can be gradually reduced to one treatment (drug+UV light exposure) every 4 to 6 weeks to maintain remission. CR has been reported in up to 71.4% of patients with early-stage MF. PUVA is less effective in tumor stage/erythrodermic and folliculotropic MF. Common PUVA side effects include erythema, photodermatitis, pruritus, and nausea, managed with dose reduction/interruption. Repeated PUVA use is limited due to the significant risk of secondary melanoma, photoaging and skin damage. PUVA carries a black-box warning for the risk of melanoma and cataracts.

Phototherapy: Ultraviolet B light (UVB) also suppresses neoplastic T cell proliferation most likely through the induction of tumor cell apoptosis. Narrowband (NB) UVB (311 nm) is used more frequently in early-stage CTCL, despite its lower efficacy, due to its lesser carcinogenic effect relative to PUVA; however, there are also increased rates of skin cancer with NB UVB as well as with PUVA. In stage IA/IB MF and parapsoriasis, CRR ranged from 54.2% to 91%, with a higher efficacy in patch compared to plaque disease. Photoaging and photo-carcinogenesis are long-term risks of NB UVB.

The above-described therapies for early-stage CTCL have serious limitations including, but not limited to, the following:

(1) Long-term use of topical steroids can lead to profound skin atrophy and the negative sequelae of systemic steroid absorption. Efficacy also rapidly wanes, with most diagnosed CTCL patients being refractory to topical steroid use.

(2) Topical or systemic retinoids, such as bexarotene, give varying clinical responses, react by unknown mechanisms, can be expensive, can cause profound skin irritation, and can have other systemic complications. The oral form is limited by systemic side effects including hyperlipidemia, pancreatitis, and birth defects.

(3) Topical mechlorethamine is somewhat effective in early-stage CTCL and is a potent alkylating agent leading to profound skin hypersensitivity reactions in a high percentage of patients and subsequent discontinuance of use. Use of topical mechlorethamine is also associated with social isolation and intimacy issues.

(4) PUVA is not approved for CTCL patients. Having previously being considered a relatively harmless form of light radiation, UVA has now been associated with deleterious effects on DNA similar to those caused by UVB with an associated increased risk of developing other skin cancers. Psoralen can be given topically or orally but the largest use is by the oral route of administration. Patients therefore must limit their exposure to sunlight, which can be a severe inconvenience. Importantly, in addition to the common side effects noted above, psoralen is also mutagenic and can cause photodamage, squamous cell carcinoma, and the risk of malignant melanoma, which can increase with chronic usage.

(5) Phototherapy, including NB UVB, has somewhat lower efficacy than photodynamic therapy like PUVA, but is still associated with increased risks of photodamage including photo-carcinogenesis and photoaging. NB UVB is also considered to be less efficacious against plaques and thicker and/or deeper lesions (e.g., folliculotropic CTCL).

It is understood in the art that current management of early-stage CTCL is inadequate and places patients at risk of short-term and long-term toxicities, some of which can themselves be both seriously debilitating and life-threatening. Patients often cycle through multiple therapies due to toxicities, even with currently available skin-directed therapies.

Watchful waiting, which accounts for between 7-14% of patients (Quaglino et al, 2012; Quaglino et al, 2021), is most likely due to a reluctance in exposing the patient to the risks and side effects of currently available treatments including prolonged topical corticosteroid or chemotherapy use or treatment with PUVA. This cautious approach to treating early-stage CTCL could place the patient at risk of an earlier and more accelerated progression to a more severe stage of disease with poorer prognosis (survival rates decrease as the level of cutaneous involvement increases).

In summary, patients diagnosed with early stages of disease are predominantly managed with skin directed therapies including topical corticosteroids, chemotherapy creams (chlormethine, mechlorethamine [Ledaga®]), phototherapy (UV treatments, such as PUVA) or radiotherapy (either local conventional radiotherapy or TSEBT). No specific photodynamic therapy or phototherapy, although used quite extensively, is approved for CTCL. Patients diagnosed with early-stage MF may also be monitored through watchful waiting where the disease is actively monitored without any lymphoma specific treatment. In that case, the patient will have regular check-ups with a specialist and treatment will be restricted to the use of a moisturizing cream and bath emollients to reduce symptoms. This approach largely reflects the limitations and potential toxicities of the available methods and treatments, and highlights the need for safe and efficacious therapies for early-stage CTCL.

There remains a need in the art to have a stable form of composition, combined with photodynamic therapy, in order to treat CTCL.

### SUMMARY OF THE INVENTION

The present invention provides for a method of treating CTCL comprising applying a combination of an effective amount of hypericin together with a form of visible light photodynamic therapy. Preferably, the effective amount of hypericin is an ointment comprising less than 1% hypericin. More preferably, the form of photodynamic therapy comprises an administration of escalating doses of visible light. Optionally, the escalating doses of visible light starts at about 5 J/cm² and increases to a maximum dose of about 12 J/cm².

In one aspect, the escalating doses of visible light is increased by about 1 J/cm² from about 1 week to about 3 weeks. Optionally, the escalating doses of visible light will continue until about 12 J/cm² or until light erythema of the lesions is observed.

In one aspect, the administration of hypericin ointment is administered at least once weekly. In another aspect, the administration of hypericin ointment is administered at least twice weekly. Application of the ointment is followed by 12-24 hours of absorption into the skin, while covered, and then followed by a light exposure.

In another aspect, application of hypericin ointment and light treatment is continued twice weekly for at least 6 weeks.

In another embodiment, the effective amount of hypericin is administered more frequently than the form of visible light photodynamic therapy. In an alternative embodiment, the effective amount of hypericin is administered less frequently than the form of visible light photodynamic therapy.

### BRIEF DESCRIPTION OF THE DRAWINGS

The following figures provide illustrative examples of the present invention and are incorporated by reference within this disclosure.

FIG. 1 shows an outline of the Phase III study supporting the preferred embodiment of the present invention.

FIG. 2 shows patient success rates over time (Cumulative Composite Assessment of Index Lesion Severity (CAILS)≥50% Improvement from Baseline) Over Time (ITT).

### DETAILED DESCRIPTION OF THE INVENTION

Topically applied hypericin, combined with ordinary fluorescent light, has the potential to revolutionize the treatment of early-stage CTCL. In a preferred embodiment, the present invention provides for a photodynamic therapy for CTCL without using potentially cancer-causing UV light.

### Examples

The following examples illustrate the various embodiments of the present invention and are not meant to be limiting in scope based on such examples.

### A. Phase III Study: Study Design

This study was a multi-center, Phase 3 double-blind, placebo-controlled, randomized study in CTCL in which 169 subjects were enrolled across 37 study centers in the United States. 166 subjects received study drug and formed the Intent to Treat (ITT) population. The study assessed the efficacy and safety of 0.25% topical hypericin under occlusion for 18-24 hours followed by administration of escalating visible light doses starting at 5 J/cm2 and increasing to maximum tolerability (≤12 J/cm2), evaluated in up to 3 cycles of treatment.

Patients meeting the inclusion criteria and none of the exclusion criteria, were randomized 2:1 to receive 0.25% ointment of the preferred embodiment of the present invention (HyB) or a placebo-matched ointment. The two treatment arms were balanced with regards to their baseline characteristics.

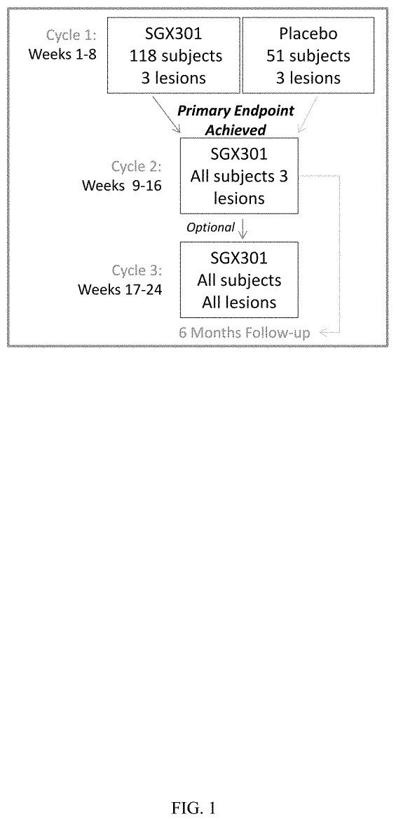

All patients received visible light treatments, starting at 5 J/cm2 and could be increased by the investigator by 1 J/cm2 at each biweekly visit until light erythema was observed (maximum light dose was 12 J/cm2). Treatment in each cycle was undertaken for 6 weeks, and then the lesion scores were again assessed after a 2-week rest period to allow the light induced erythema to fade and the maximal lesion effects to become evident. Cycles 1 (blinded) and 2 (cross-over design with all subjects receiving HyB) were both initiated at the 5 J/cm2 light dose level. Cycle 3 was optional and considered a compassionate use type of cycle, where patients could treat all of their lesions (as opposed to the 3 index lesions treated during Cycles 1 and 2), and the light dose was continued from the maximum value obtained in Cycle 1 and/or 2. Evaluation of safety and treatment response during the open-label cycle took place at Week 24 following a 2-week rest period to permit any light induced erythema to subside (FIG. 1).

Key demographics for the study population are available in Table 1 and Table 2, as further evidenced below:

TABLE 1
Demographics and Baseline Characteristics (ITT Population)

| | HyB | | Placebo | | Total | | Comparison |
|---|---|---|---|---|---|---|---|
| Characteristic | n = 116 | | n = 50 | | n = 166 | | p-value |
| Gender, n (%) | | | | | | | 0.5117 ^{a} |
| Male | 69 | (59.5) | 27 | (54.0) | 96 | (57.8) | |
| Female | 47 | (40.5) | 23 | (46.0) | 70 | (42.2) | |
| Race, n (%) | | | | | | | 0.1466 ^{a} |
| White | 84 | (72.4) | 36 | (72.0) | 120 | (72.3) | |
| Black | 27 | (23.3) | 12 | (24.0) | 39 | (23.5) | |
| Asian | 4 | (3.4) | 0 | (0.0) | 4 | (2.4) | |
| Native Hawaiian/Pacific Islander | 0 | (0.0) | 0 | (0.0) | 0 | (0.0) | |
| American Indian/Alaska Native | 0 | (0.0) | 0 | (0.0) | 0 | (0.0) | |
| Other | 0 | (0.0) | 2 | (4.0) | 2 | (1.2) | |
| Unknown | 1 | (0.9) | 0 | (9.0) | 1 | (0.6) | |
| Ethnicity, n (%) | | | | | | | 0.7619 ^{a} |
| Hispanic or Latino | 10 | (8.6) | 3 | (6.0) | 13 | (7.8) | |
| Not Hispanic or Latino | 103 | (88.8) | 45 | (90.0) | 148 | (89.2) | |
| Not Reported | 3 | (2.6) | 2 | (4.0) | 5 | (3.0) | |
| Age, (years) ^{c} | | | | | | | 0.5970 ^{b} |
| Mean (SD) | 57.9 | (15.94) | 59.4 | (16.26) | 58.4 | (16.00) | |
| Median | 59.0 | | 62.5 | | 60.0 | | |
| Range | 19, | 99 | 20, | 89 | 19, | 99 | |
| Height, cm | | | | | | | 0.1524 ^{b} |
| n | 113 | | 47 | | 160 | | |
| Mean (SD) | 170.85 | (11.644) | 173.61 | (9.537) | 171.66 | (11.109) | |
| Median | 172.70 | | 175.00 | | 172.70 | | |
| Range | 132.1, | 196.8 | 152.4, | 200.7 | 132.1, | 200.7 | |
| Weight, kg (SD)1 | | | | | | | 0.3547 ^{b} |
| Mean (SD) | 88.18 | (23.209) | 91.57 | (17.359) | 89.20 | (21.619) | |
| Median | 86.15 | | 90.30 | | 86.95 | | |
| Range | 43.9, | 201.8 | 54.4, | 123.5 | (43.9, | 201.8) | |
| Body Mass Index ^{d} | | | | | | | 0.9721 ^{a} |
| n | 113 | | 47 | | 160 | | |
| Mean (SD) | 30.21 | (6.989) | 30.26 | (5.714) | 30.23 | (6.623) | |
| Median | 28.80 | | 28.32 | | 28.80 | | |
| Range | 19.3, | 60.3 | 21.3, | 44.0 | 19.3, | 60.3 | |

TABLE 1
Baseline CTCL Characteristics (ITT Population)

| | HyB | | Placebo | | Total | | Comparison |
|---|---|---|---|---|---|---|---|
| Characteristic | n = 116 | | n = 50 | | n = 166 | | p-value |
| Stage, n (%) | | | | | | | 0.3093 ^{a} |
| n | 116 | | 50 | | 166 | | |
| IA | 72 | (62.1) | 31 | (62.0) | 103 | (62.0) | |
| IB | 39 | (33.6) | 19 | (38.0) | 58 | (34.9) | |
| IIA | 5 | (4.3) | 0 | (0.0) | 5 | (3.0) | |
| Duration of disease ^{b}, months | | | | | | | 0.6435 ^{c} |
| n | 116 | | 50 | | 166 | | |
| Mean (SD) | 75.84 | (78.885) | 82.33 | (91.157) | 77.80 | (82.546) | |
| Median | 51.36 | | 38.55 | | 49.39 | | |
| Range | 0.4, | 375.6 | 0.5, | 396.1 | 0.4, | 396.1 | |
| Baseline CAILS score of Index Lesions | | | | | | | 0.9967 ^{c} |
| n | 116 | | 50 | | 166 | | |
| Mean (SD) | 32.6 | (13.69) | 32.6 | (12.49) | 32.6 | (13.30) | |
| Median | 30.0 | | 32.5 | | 30.0 | | |
| Range | 8, | 73 | 3, | 55 | 3, | 73 | |
| Number of previous other CTCL therapies | | | | | | | 0.0803 ^{c} |
| n | 116 | | 50 | | 166 | | |
| Mean (SD) | 3.9 | (3.65) | 2.9 | (2.47) | 3.6 | (3.36) | |
| Median | 3.0 | | 2.0 | | 2.5 | | |
| Range | 0, | 19 | 0, | 12 | 0, | 19 | |

^{a} p-value from chi-squared test1
^{b} Defined as time of first reported diagnosis of CTCL to randomization
^{c} p-value from student's t-test

Only two patients were discontinued for adverse events. Both patients had unacceptable burning sensations with light therapy while receiving HyB treatment (1.7%) and were withdrawn from the study. None of the placebo patients who received HyB in Cycle 2 were withdrawn for burning (total withdrawn receiving HyB, 1.2%).

The primary efficacy endpoint for this trial was the proportion of patients achieving a treatment response, defined as a Composite Assessment of Index Lesion Severity (CAILS) score ratio comparing the CAILS score at the end of Cycle 1 (Week 8) assessment divided by the CAILS score at baseline of ≤50% (≥50% reduction in the CAILS score) of the cumulative total score of the prospectively selected 3 treated index lesions. During the observational, Cycle 2 portion of the study, a similar secondary endpoint for successful treatment was used (≥50% improvement in the cumulative CAILS score of the 3 index lesions at the end of Cycle 2 compared to baseline). The CAILS score is a standard measurement of lesion severity in CTCL studies and is calculated by assessing the erythema score, scaling score, plaque elevation score and involved surface area score for each of the index lesions. Each of the assessments and the total score for each evaluated lesion was recorded in the clinical report form (CRF). The total CAILS score was calculated by adding the scores of all evaluated lesions together.

Key secondary endpoint measures include assessment of response duration, degree of improvement, time to relapse and safety.

### B. Phase II Study: Efficacy Results

A patient was considered a responder if their cumulative CAILS (Olsen et al, 2011) score summed over the 3 index lesions was decreased by at least 50% relative to baseline. At the end of Cycle 1, 16.4% of patients were considered responders (versus 4% placebo group; p=0.04) with 2 complete responders (Table 3).

TABLE 3
HyB Response Rate by Treatment Group and Cycle

| | Placebo | HyB | HyB | HyB |
|---|---|---|---|---|
| | Response Rate | Response Rate | Response Rate | Response Rate |
| Parameter | (End Cycle 1) | (End Cycle 1) | (End Cycle 2) | (End Cycle 3) |
| Overall | Placebo: | HyB: | Placebo + HyB: | HyB × 3 Cycles: |
| Response | 2/50 (4.0%) | 19/116 (16.4%) | 10/45 (22.2%) | 38/79 (48.7%) |
| | | p = 0.0416 vs | P = 0.0047 vs | P < 0.0001 vs |
| | | Placebo Cycle 1 | Placebo End Cycle 1 | Cycle 1 placebo |
| | | | HyB + HyB: | |
| | | | 44/110 (40.0%) | |
| | | | p < 0.0001 vs | |
| | | | Placebo End Cycle 1 | |
| | | | p < 0.0001 vs | |
| | | | HyB End Cycle 1 | |

Response was found to be considerably improved following more prolonged treatment with 40% of the 110 patients receiving HyB throughout both Cycles 1 and 2 judged to be responders ([p<0.0001 versus placebo) (Table 1), with 7 complete responders. This represents an increase from 15.5% to 40% response in the 110 patients who continued treatment with HyB through Cycle 2. It was notable that there was a 22.2% response rate in patients who switched from placebo in Cycle 1 to HyB in Cycle 2, thereby demonstrating consistency of the rapid response observed in Cycle 1 patients treated with HyB.

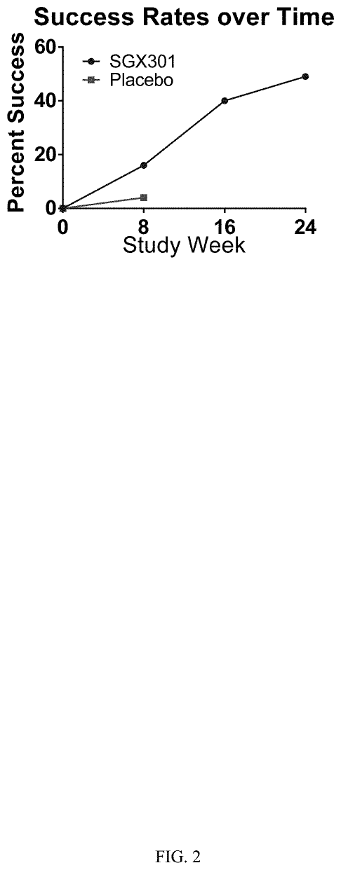

Treatment was continued throughout Cycle 3 (open label compassionate use) on an optional basis to assess impacts of long-term treatment. A further increase in treatment benefit was observed in the 78 patients who received all 3 Cycles, with response rate increasing from 40% at Cycle 2 to 48.7% following Cycle 3 in these patients. However, it should be noted that the Cycle 3 results represent a selection bias since not all patients opted to continue. The increase in response rate over time is clearly illustrated at FIG. 2.

The rapid response rate (16.4% over 6 weeks) is coupled with a broader response including an average improvement in cumulative CAILS score of 24% (across all subjects in Cycle 1, indicating that many patients were seeing improvement in their lesions even if it did not meet the definition of “success”). The average reduction in the CAILS lesion score increased to 37% over baseline following Cycle 2. This rapid response enables rapid decision making regarding continuing care with HyB versus trying alternative therapies and differentiates HyB from other therapies, which often require many months to show benefit.

Treatment response to HyB was similar across the patient population (Table 4) albeit not statistically significant due to the small sample size. Treatment was also similarly effective irrespective of the previous duration of disease or the number of prior therapies utilized (Table 5). The variable range in previous duration of disease and number of prior treatments are both representative of the overall clinical population. Thus, HyB offers potential skin-directed treatment for patients with early-stage disease, irrespective of the previous patient journey and with a very low adverse event rate.

TABLE 2
HyB and Placebo Patient Response Rates across Patient
Demographics and Baseline Disease Characteristics

| | Week 8 Response Rate | | |
|---|---|---|---|
| Subpopulation | HyB | Placebo | p-value ^{a} |
| All (primary endpoint) | 16.4% (n = 116) | 4.0% (n = 50) | 0.0416 |
| White (race) | 17.9% (n = 84) | 5.6 (n = 36) | 0.0858 |
| Black (race) | 11.1% (n = 37 | 0% (n = 12) | — |
| Maximum light ≤10 J | 17.6% (n = 51) | 5.9% (n = 17) | 0.2600 |
| Maximum light >10 J | 15.4% (n = 65) | 3.0% (n = 33) | 0.0945 |
| Tumor stage IA | 18.1% (n = 72) | 3.2% (n = 31) | 0.0688 |
| Tumor stage IB | 12.8% (n = 39) | 5.3% (n = 19) | 0.6757 |
| Male | 14.5% (n = 69) | 3.7% (n = 27_{—} | 0.1655 |
| Female | 19.1% (n = 47) | 4.3% (n = 23) | 0.1329 |
| ≤60 years old | 16.9% (n = 65) | 4.5% (n = 22) | 0.3516 |
| >60 years old | 15.7% (n = 51) | 3.6% (n = 28) | 0.1514 |

TABLE 3
Treatment Response as a Function of the Number of Prior Therapies ^{a}

| Number | | | | | | | | | |
|---|---|---|---|---|---|---|---|---|---|
| of Prior | Week 8 Response Rate | | | Week 16 Response Rate | | | Week 24 Response Rate | | |
| Therapies | HyB | Placebo | p-value | HyB | Placebo | p-value | HyB | Placebo | p-value |
| Any | 16.4% | 4.0% | 0.0416 | 40.0% | 4.0% | <0.0001 | 48.7% | 4.0% | <0.0001 |
| | (n = 116) | (n = 50) | | (n = 110) | (n = 50) | | (n = 78) | (n = 50) | |
| ≤1 | 16.7% | 6.7 | 0.4029 | 42.4% | 6.7 | 0.018 | 57.7% | 6.7 | 0.0021 |
| | (n = 36) | (n = 15) | | (n = 33) | (n = 15) | | (n = 26) | (n = 15) | |
| ≥2 | 16.3% | 2.9 | 0.0598 | 39.0% | 2.9 | <0.0001 | 44.2% | 2.9 | <0.0001 |
| | (n = 80) | (n = 35) | | (n = 77) | (n = 35) | | (n = 52) | (n = 35) | |
| 2 or 3 | 18.2% | 4.3% | 0.1756 | 48.4% | 4.3% | 0.0006 | 45.5% | 4.3% | 0.0117 |
| | (n = 33) | (n = 23) | | (n = 31) | (n = 23) | | (n = 22) | (n = 23) | |
| >3 | 14.9 | 0% | — | 32.6% | 0% | 0.0251 | 43.3% | 0% | 0.0077 |
| | (n = 47) | (n = 12) | | (n = 46) | (n = 12) | | (n = 30) | (n = 12) | |

^{a} The analysis was based on a logistic regression model with treatment and baseline total CAILS score as independent variables.

Duration of treatment response was also sustained throughout the follow-up period in 53% of patients, with an undefined median response. Logistic regression analysis indicated that the duration of response was statistically significantly correlated with the number of cycles of HyB therapy, with patients with more cycles of therapy having a more durable response (p=0.0128).

Efficacy responses could also be observed on a per lesion basis and moreover, lesions could be characterized by their thickness, representing the occurrence of both patches (thickness=0) and plaques (thickness>0) in this early-stage patient population. The results of this analysis indicated that HyB is effective against both patches and plaques (Table 6), another differentiating feature since other skin directed therapies (e.g., NB UVB) are known to be effective primarily against patches. Thus, the results suggest that HyB is at least as effective in the thicker plaque lesions as it is in the thinner patch lesions allowing a broader application of the treatment to a wider range of disease than is available with UV therapies which are limited by having less depth of skin penetration.

TABLE 4
HyB Response Rate by Lesion

| | Placebo | | HyB | HyB |
|---|---|---|---|---|
| | Response Rate | | Response Rate | Response Rate |
| Parameter | (End Cycle 1) | | (End Cycle 1) | (End Cycle 2) |
| Overall Lesion | 21/150 | (14%) | 74/348 (21%) | HyB + HyB: 129/330 (39%) |
| Efficacy | | | p = 0.06 vs | p < 0.0001 vs Placebo End Cycle 1 |
| | | | Placebo Cycle 1 | p < 0.0001 vs HyB End Cycle 1 |
| Patch Lesions | 14/85 | (17%) | 33/184 (18%) | HyB + HyB: 65/178 (37%) |
| | | | p = 0.77 vs | P = 0.0009 vs Placebo End Cycle 1 |
| | | | Placebo Cycle 1 | p < 0.0001 vs HyB End Cycle 1 |
| Plaque Lesions | 7/65 | (11%) | 41/264 (25%) | HyB + HyB: 64/152 (42%) |
| | | | p = 0.01 vs | p < 0.0001 vs Placebo End Cycle 1 |
| | | | Placebo Cycle 1 | p < 0.0001 vs HyB End Cycle 1 |

It was noteworthy that efficacy was also observed in patients with more difficult to treat CTCL, including folliculotropic MF. In this more aggressive form of MF, malignant cells present deeper in the skin surrounding hair follicles (Mehta-Shah et al, 2020). Hypericin has maximal absorption between 500 and 650 nm in wavelength and this part of the visible spectrum is known to penetrate significantly deeper than ultraviolet light (Gökdemir et al, 2006; Ash et al, 2017).

Current recommendations from the National Comprehensive Cancer Network Guidelines (NCCN) for the treatment of folliculotropic MF are to move to systemic agents due to current lack of efficacy of available skin-directed therapies (Mehta-Shah et al, 2020). The absorption spectrum of hypericin and the clinical response observed in the current study suggest HyB will provide an alternative therapy for some patients with this variant of CTCL. This may allow some patients to avoid costlier and dangerous systemic therapies. Indeed, it was shown at the end of Cycle 2 treatment with HyB, that all types of lesions demonstrated a statistically significant, and similar, response rate (see Table 6) This was an unexpected response given the previously reported reduced efficacy of hypericin in ointment form in thicker psoriasis lesions (Rook et al.).

During Cycle 3, plasma samples were obtained in the first 29 patients within 30 minutes of completing each of the two light treatment sessions during Week 5 and Week 6 of Cycle 3 to determine the concentration of hypericin in the circulating plasma following topical application of HyB to all lesions. As patients were treating more body surface area in this Cycle, it was expected that any systemic absorption would be highest in this treatment period and at the end of this cycle, hypericin serum concentrations were obtained. Circulating plasma levels were undetectable in all instances. The bioanalytical method had a lower limit of quantitation of 0.005 μg/ml (or 5 ng/ml). Thus, evidence suggests that HyB represents a targeted light therapy using visible light combined with a drug substance, which is preferentially absorbed by malignant T-cells and has been shown to have no detectable circulating levels after topical treatment. This is the first demonstration of the lack of circulating hypericin after topical administration.

### C. Phase II Study: Safety Profile

The safety profile to date has been benign with the most common adverse event (AE) being related to the application site burning/tingling. This type of AE is common in CTCL and the incidence rate and severity with HyB is significantly less than other treatment modalities (e.g., mechlorethamine).

There were 8 severe AEs reported from 7 patients in the trial. There was no apparent consistent increase in severe AEs reported in the HyB group compared to placebo other than drug site application pain and erythema. AEs considered possibly related (occurring in 11% of patients receiving HyB) or related (occurring in 15% of patients receiving HyB) to study drug predominantly included skin reactions including pruritus, hyperpigmentation, burning, pain and irritation and general administrative procedures like application site pain and pruritus.

There were 5 serious adverse events (SAEs) during the study, one of which occurred in a patient receiving placebo Cycle 1 treatment. Of the remaining 4 SAEs, 2 occurred greater than 2 weeks after the most recent topical HyB treatment. The remaining 2 SAEs occurred during Cycle 3 in patients that had received placebo in Cycle 1, HyB in Cycle 2 and elected to continue to receive HyB in Cycle 3. Of the 5 SAEs, 4 were considered to be related to underlying medical conditions or medications that were unrelated to CTCL. All of the SAEs resolved, and none were considered related to HyB treatment. There was no consistent pattern in the SAEs, nor did the SAEs occur in categories that were highly represented in the study-reported AEs, suggesting they were sporadic and unrelated events.

There were no deaths during the study. There were no clinically meaningful changes in any hematology or clinical chemistry parameters. While there were potentially significant changes in vital signs recorded in some patients, they were similarly distributed in both an upward (e.g., increased blood pressure) and downward (e.g., decreased blood pressure) direction with no obvious correlation to study drug.

The encouraging safety profile of HyB and visible light is in stark contrast to the application of some standard therapies currently used to treat early stages of CTCL such as topical mechlorethamine or oral psoralen with UVA, which are associated with mutagenesis leading to a risk of melanoma and non-melanoma skin cancers (Nijsten et al, 2003; Vonderheid et al, 1989).

The incidence of AEs by System Organ Class (SOC) and preferred term is summarized in Table 7 for each of the treatment periods where incidence >3%. TEAEs were reported in 69.9% of patients overall, and 67.1% of patients who received HyB. The most common TEAEs reported in patients who received HyB were in the SOCs of Skin and Subcutaneous Tissue Disorders (29.8%), Infections and Infestations (26.7%), and General Disorders and Administration Site Conditions (23.6%).

TABLE 5
Adverse Events by System Organ Class and Preferred Term, Incidence ≥3%

| | Cycle 1 | | | | Cycle 2 | | Cycle 3 | | HyB | | | |
|---|---|---|---|---|---|---|---|---|---|---|---|---|
| | HyB | | Placebo | | HyB | | HyB | | Total | | Total | |
| | (N = 116) | | (N = 50) | | (N = 155) | | (N = 110) | | (N = 161) | | (N = 166) | |
| Preferred Term | n (%) | | n (%) | | n (%) | | n (%) | | n (%) | | n (%) | |
| #Patients ≥1 TEAE | 56 | (48.3) | 27 | (54.0) | 66 | (42.6) | 49 | (44.5) | 108 | (67.1) | 116 | (69.9) |
| Skin and subcutaneous tissue | 19 | (16.4) | 5 | (10.0) | 21 | (13.5) | 19 | (17.3) | 48 | (29.8) | 53 | (31.9) |
| disorders | | | | | | | | | | | | |
| Pruritus | 6 | (5.2) | 2 | (4.0) | 2 | (1.3) | 5 | (4.5) | 12 | (7.5) | 14 | (8.4) |
| Erythema | 3 | (2.6) | 0 | | 3 | (1.9) | 1 | (0.9) | 7 | (4.3) | 7 | (4.2) |
| Infections and infestations | 20 | (17.2) | 10 | (20.0) | 19 | (12.3) | 16 | (14.5) | 43 | (26.7) | 51 | (30.7) |
| Upper respiratory tract | 8 | (6.9) | 4 | (8.0) | 2 | (1.3) | 2 | (1.8) | 12 | (7.5) | 16 | (9.6) |
| infection | | | | | | | | | | | | |
| Viral upper respiratory tract | 4 | (3.4) | 0 | | 4 | (2.6) | 4 | (3.6) | 11 | (6.8) | 11 | (6.6) |
| infection | | | | | | | | | | | | |
| Sinusitis | 1 | (0.9) | 0 | | 3 | (1.9) | 3 | (2.7) | 7 | (4.3) | 7 | (4.2) |
| Urinary tract infection | 1 | (0.9) | 2 | (4.0) | 3 | (1.9) | 0 | | 4 | (2.5) | 6 | (3.6) |
| Influenza | 0 | | 2 | (4.0) | 1 | (0.6) | 2 | (1.8) | 3 | (1.9) | 5 | (3.0) |
| General disorders and | 22 | (19.0) | 5 | (10.0) | 18 | (11.6) | 12 | (10.9) | 38 | (23.6) | 42 | (25.3) |
| administration site conditions | | | | | | | | | | | | |
| Application site pain | 8 | (6.9) | 2 | (4.0) | 5 | (3.2) | 6 | (5.5) | 16 | (9.9) | 17 | (10.2) |
| Application site pruritus | 5 | (4.3) | 1 | (2.0) | 5 | (3.2) | 0 | | 9 | (5.6) | 9 | (5.4) |
| Fatigue | 3 | (2.6) | 1 | (2.0) | 3 | (1.9) | 2 | (1.8) | 8 | (5.0) | 9 | (5.4) |
| Application site paraesthesia | 6 | (5.2) | 0 | | 2 | (1.3) | 2 | (1.8) | 7 | (4.3) | 7 | (4.2) |
| Pain | 3 | (2.6) | 0 | | 2 | (1.3) | 0 | | 5 | (3.1) | 5 | (3.0) |
| Nervous system disorders | 9 | (7.8) | 4 | (8.0) | 6 | (3.9) | 5 | (4.5) | 18 | (11.2) | 22 | (13.3) |
| Headache | 5 | (4.3) | 3 | (6.0) | 5 | (3.2) | 2 | (1.8) | 10 | (6.2) | 13 | (7.8) |
| Dizziness | 3 | (2.6) | 1 | (2.0) | 1 | (0.6) | 2 | (1.8) | 6 | (3.7) | 7 | (4.2) |
| Hyperaesthesia | 0 | | 1 | (2.0) | 0 | | 1 | (0.9) | 1 | (0.6) | 2 | (1.2) |
| Migraine | 0 | | 0 | | 0 | | 1 | (0.9) | 1 | (0.6) | 1 | (0.6) |
| Paraesthesia | 1 | (0.9) | 0 | | 0 | | 0 | | 1 | (0.6) | 1 | (0.6) |
| Presyncope | 0 | | 0 | | 0 | | 1 | (0.9) | 1 | (0.6) | 1 | (0.6) |
| Syncope | 0 | | 1 | (2.0) | 0 | | 0 | | 0 | | 1 | (0.6) |
| Respiratory, thoracic and | 9 | (7.8) | 1 | (2.0) | 5 | (3.2) | 5 | (4.5) | 17 | (10.6) | 17 | (10.2) |
| mediastinal disorders | | | | | | | | | | | | |
| Injury, poisoning and | 6 | (5.2) | 2 | (4.0) | 4 | (2.6) | 5 | (4.5) | 14 | (8.7) | 16 | (9.6) |
| procedural complications | | | | | | | | | | | | |
| Musculoskeletal and connective | 3 | (2.6) | 3 | (6.0) | 6 | (3.9) | 4 | (3.6) | 11 | (6.8) | 14 | (8.4) |
| tissue disorders | | | | | | | | | | | | |
| Neoplasms benign, malignant | 0 | | 1 | (2.0) | 3 | (1.9) | 2 | (1.8) | 5 | (3.1) | 6 | (3.6) |
| and unspecified (incl cysts and | | | | | | | | | | | | |
| polyps) | | | | | | | | | | | | |
| Eye disorders | 3 | (2.6) | 0 | | 2 | (1.3) | 0 | | 5 | (3.1) | 5 | (3.0) |
| Vascular disorders | 2 | (1.7) | 1 | (2.0) | 1 | (0.6) | 1 | (0.9) | 4 | (2.5) | 5 | (3.0) |

#= Number;
TEAE = treatment-emergent adverse event,
Note:
TEAE was defined as an AE that was new or worsened in severity after the first dose of study dmg and within 1 month following last evaluation visit. At each summary level (SOC, PT), a patient was counted only once or each AE he/she experienced within that level. All adverse events were coded using the Medical Dictionary for Regulatory Activities (MedDRA), Version 20.

TEAEs reported in ≥5% of patients who received HyB were application site pain (9.9%), pruritus (7.5%), upper respiratory tract infection (7.5%), viral upper respiratory tract infection (6.8%), headache (6.2%) application site pruritus (5.6%), and fatigue (5.0%) (Table 7).

In Cycle 1, application site events were reported in a higher proportion of patients who received HyB than in those who received placebo. These events included application site pain (6.9% versus 4.0%), application site pruritus (4.3% versus 2.0%), application site paraesthesia (5.2% versus 0), application site erythema (1.7% versus 0), application site warmth (0.9% versus 0), administration site reaction (0.9% versus 0), application site irritation (0.9% versus 0). The only application site event reported in a high proportion of patients who received placebo was application site discoloration, in 0.9% of patients versus no patient who received HyB.

The proportion of patients who reported application site reactions did not appear to increase with subsequent exposure to HyB in Cycle 2 and Cycle 3.

In summary, this study demonstrates that HyB, administered twice weekly in 6-week cycles, can:

(1) induce a rapid treatment response (within 6 weeks of treatment);

(2) achieve up to 40% treatment response within 12 weeks of treatment and 49% with 18 weeks of treatment;

(3) have similar efficacy on both patches and plaques, which both occur in early-stage CTCL;

(4) minimize exposure to circulating concentrations of hypericin; and

(5) be safe and well tolerated.

The HyB embodiment of the present invention represents a substantial improvement over the current state of the art relative to CTCL therapies. As shown in Table 8, there are great benefits in using HyB when compared to published data from the clinical trials of approved alternative therapies for early-stage CTCL. HyB showed unexpected efficacy in plaque as well as patch disease, and in variants of CTCL with deeper skin involvement. HyB is expected to provide a range of benefits over existing therapies and has therefore the potential to address an unmet need in early-stage CTCL.

TABLE 6
Comparison of HyBryte Published Results of Approved Drug Treatment in Early-Stage CTCL

| | | | | | | | | End- | Response | Response | Response | |
|---|---|---|---|---|---|---|---|---|---|---|---|---|
| Drug | Phase | Design | Disease | Dose | N | Duration | | point | 8 week | 16 Week | 24 Week | Reference |
| HyBryte | III | DB, PC | IA, IB, | 0.25% | 116 | 16-24 | weeks | CAILS | 16% | 40% | 49% | HPN- |
| Active arm | | | IIA | twice | | | | | | | | CTCL-01 |
| | | | | weekly | | | | | | | | |
| HyBryte | III | DB, PC | IA, IB, | twice | 50 | 8 | weeks | CAILS | 4% | ND | ND | HPN- |
| Placebo arm | | | IIA | weekly | | | | | | | | CTCL-01 |
| Topical | III | Obs. | IA, IB, | Titrated | 50 | ≥16 | weeks | CAILS | 21% | 41% | 49% | Heald 2003 |
| Bexarotene | | | IIA | qod to | | | | | | | | |
| gel | | | refractory | QID | | | | | | | | |
| | | | to ≥2 | | | | | | | | | |
| | | | previous | | | | | | | | | |
| | | | treatments | | | | | | | | | |
| Mechloretha | III | Non- | IA, IB, | 0.02% | 130 | 12 | months | CAILS | 0% | 20% | 36% | Lessin |
| mine (MCH) | | infer | IIA | MCH | | | | | | | | 2013 |
| gel | | | | gel | | | | | | | | |
| Mechloretha | III | Non- | IA, IB, | 0.02% | 130 | 12 | months | CAILS | 0% | 20% | 48% | Lessin |
| mine (MCH) | | infer | IIA | MCH | | | | | | | | 2013 |
| ointment | | | | ointment | | | | | | | | |
| Oral | III | Obs. | IA, IB, | | 58 | ≥16 | weeks | CAILS | | | | Duvic 2001 |
| Bexarotene | | | IIA | 6.5 | 15 | | | | NR | NR | 20%¹ | |
| | | | refractory | mg/m² | | | | | | | | |
| | | | to ≥2 | 300 | 28 | | | | NR | NR | 36%¹ | |
| | | | previous | mg/m² | | | | | | | | |
| | | | treatments | >300 | 15 | | | | NR | NR | 47%¹ | |
| | | | | mg/m² | | | | | | | | |

Abbreviations:
DB = double blind;
PC = placebo controlled;
Obs, = observational;
Non-infer = non-inferiority design;
qd = quaque die, daily;
BID = bis in die, twice a day;
QID = quater in die, four times a day;
qod = quaque alternis die; every other day;
NR = not reported
¹At end of therapy, time not stated

HyB displays comparable or greater efficacy vs other approved early-stage CTCL therapies, and achieves a more rapid response. HyB treatment efficacy and safety results are generally superior to those reported for approved therapies for the treatment of refractory early-stage CTCL using less rigorous trial designs, while being better tolerated with a lower drop-out rate.

In the placebo-controlled pivotal Phase 3 study described in the above Examples, HyB displayed a 16% response rate after 8 weeks (cycle 1; 6 weeks treatment followed by 2 week “rest period”), with prolonged therapy dramatically increasing the success rate: up to 40% response rate at 16 weeks (cycle 2, patients that received HyB during cycle 1) and 49% at 24 weeks (cycle 3, patients receiving HyB for all 3 cycles).

While comparable response rates are achieved at 24 weeks for approved therapies such as mechlorethamine or topical and oral (higher doses) bexarotene, it is worth noting that in the majority of the key studies supporting those approvals, the product benefitted from a study design with inherent patient bias due to the lack of an adequate control group (i.e. the patient was on some type of active agent and expected treatment to work). In contrast, the response rate in the HyB study was rigorously defined in a placebo-controlled setting and, owing to the logistical commitment needed by the patients (e.g., required to visit the site, often inconveniently located clinic twice per week attend for up to 24 weeks if they complete Cycle 3) to remain in the study and the possibility of receiving placebo, the full impact of the HyB after 6 weeks of therapy is likely understated. The use of a placebo-controlled study enhances the confidence of patients and physicians in assessing the utility of the treatment.

Importantly, no prior therapeutics have demonstrated efficacy with a similar or better response rate (i.e., 16%) over such a short interval of time (i.e., 6 weeks of treatment) in a placebo-controlled clinical trial. Mechlorethamine gel (non-inferiority trial between pharmacy prepared and centrally prepared formulations of mechlorethamine) did not show responses until at least 8 weeks of continuous once daily treatment and took almost 13 weeks of continuous treatment to reach a 16% response rate (Lessin et al, 2013). At 16 weeks, the response rate was 20% vs. 40% for HyB. Bexarotene gel also did not achieve 16% until at least day 60 (9 weeks of continuous treatment) (Heald et al, 2003).

This rapid response profile is extremely meaningful for the early-stage CTCL population. Treating physicians typically wait months to be able to evaluate patients' response to treatments and to conclude on the risk-benefit ratio of continuing therapy versus transitioning to another treatment modality. Having earlier time points that are significant provides the physician with more power to make these decisions earlier in the treatment process.

HyB is safe and displays significantly better tolerability vs existing treatments. The combination of targeted therapy with targeted light therapy using visible red-yellow spectrum light yields a very benign safety profile. Hypericin is not mutagenic and even when administered intravenously at much higher doses, does not cause significant AEs other than those related to photoactivity (Gulick et al, 1999; Jacobson et al, 2001). Thus, the compound itself is benign. Importantly, even after up to 18 weeks (36 applications) of treatment, over multiple body regions, no systemic absorption of hypericin is observed in the blood. This is in stark contrast to standard therapies such as mechlorethamine or PUVA which are associated with mutagenesis leading to a risk of melanoma and non-melanoma skin cancers.

When comparing HyB safety data to available data for other skin-directed therapies (Table 9), the rate of events leading to study discontinuation was markedly lower and, overall, HyB therapy has less cutaneous AEs compared to bexarotene gel or mechlorethamine gel.

TABLE 7
Reported Side Effects of HyB and other Approved Skin-Directed Therapies

| | | | Dropouts | Overall | | |
|---|---|---|---|---|---|---|
| Drug | Disease | Size | “Early” | Dropouts | Related Side effects | Ref |
| HyB drug | IA, IB, IIA | 116 | 5% | | Severe AEs: 7% (1 related | This Study |
| | | | | | to drug): | |
| | | | | | Abdominal pain | |
| | | | | | Gallbladder obstruction | |
| | | | | | Site pain during light | |
| | | | | | therapy | |
| | | | | | Severe erythema | |
| | | | | | Invasive ductal breast | |
| | | | | | carcinoma | |
| HyB placebo | IA, IB, IIA | 50 | 10% | | Severe AEs: 2% | |
| | | | | | (angioedema) | |
| Topical | IA, IB, IIA | 50 | 16% | 94% | 26% (moderate to severe) | Heald 2003 |
| Bexarotene | refractory to ≥2 | | | | Irritant dermatitis: | |
| Gel | other Rx | | | | Rash 74% | |
| | | | | | Pain 32% | |
| | | | | | Skin disorder 26% | |
| | | | | | Contact dermatitis 14% | |
| | | | | | AEs requiring dose | |
| | | | | | adjustment: 64% | |
| | | | | | AEs leading to | |
| | | | | | discontinuation: | |
| | | | | | Directly: 6% | |
| | | | | | Indirectly: 10% | |
| | | | | | Hematologic AEs: | |
| | | | | | Lymphocy1topenia 11% | |
| | | | | | Granulocytopenia: 4% | |
| | | | | | Low CD4+ counts: 16% | |
| | | | | | Hypertriglyceridemia: 4% | |
| Mechloretha | | 130 | NR | 17% | Allergic contact dermatitis: | Lessin 2013 |
| mine (MCH) | | | | | 16% | |
| ointment | | | | | Local ‘related’ skin AEs: | |
| | | | | | 62% | |
| Mechloretha | IA, IB, IIA | 130 | NR | 20% | Allergic contact dermatitis: | Lessin 2013 |
| mine (MCH) | | | | | 13% | |
| ointment | | | | | Local relate skin AEs: 50% | |
| Oral | IA, IB, IIA | 58 | NR | 31% | Hyperlipidemia: 79% | Duvic 2001 |
| bexarotene | refractory to | | | | Hypercholesterolemia: 48% | |
| | other Rx | | | | Headache: 38% | |
| | | | | | Treated for hypothyroidism: | |
| | | | | | 41% | |
| | | | | | Asthenia: 33% | |
| | | | | | Leukopenia: 28% | |
| | | | | | Pruritus: 21% | |

Abbreviations:
NR = Not Reported;

The rate of severe AEs with HyB was 4% (7 of the 161 HyB treated patients) with only 1 related to study drug. In contrast, topical bexarotene was associated with a 24-26% rate of treatment limiting, moderate to severe toxic events while topical mechlorethamine recorded >10% rate of contact allergic dermatitis and >50% rate of local skin reactions. Oral bexarotene was associated with a number of additional systemic side effects, including hyperlipidaemia and hypothyroidism as well as an elevated rate of skin-related issues including pruritus, rash and other skin disorders. Mechlorethamine treatment also created major intimacy issues for patients since treated areas of skin cannot be in contact with other people. The dropout rate from all 3 therapies was significantly higher than that seen in the HyB Phase III trial.

In summary, the in vitro data indicating inhibition of cell proliferation and apoptotic death by light-activated hypericin in lymphoid cells, taken together with the existing Phase 1, Phase 2 and Phase 3 clinical results, demonstrate cutaneous photosensitivity reactions to topically-applied, light-activated hypericin, and clearing of active skin lesions of CTCL patients, including plaques and difficult-to-treat folliculotropic lesions. The highly encouraging and statistically significant efficacy data from the randomized, placebo-controlled Phase 3 trial in CTCL described in the above Examples, coupled with the benign safety profile of hypericin and visible light, provides evidence that targeting skin lesions with light-activated hypericin can be an effective treatment strategy in CTCL patients, with the potential for improved safety and tolerability over existing options.

The overall benefits from the study results described above show the following improvements over the existing art of record:
1. Treatment response within 6 weeks of 16%;
2. Enhanced treatment response with increased treatment time—40% at 12 weeks, 49% at 18 weeks;
3. Treatment works irrespective of the length of time the patient has had disease, as long as skin-directed therapy is still relevant;
4. Treatment works irrespective of the number of prior therapies a patient has tried;
5. Treatment responses are more durable the longer the patient treats for—so longer treatment is correlated to a longer time before the lesion dramatically increases again;
6. Treatment is equally effective against both patches and plaques. This is unexpected as previous studies suggested the ointment was less effective against the thicker lesions in psoriasis;
7. Specific treatment regimen was twice weekly, with opportunities to treat for longer durations;
8. Hypericin ointment resulted in a very low dropout rate during treatment (5%) —significantly lower than other skin-directed therapies.

As used in this specification and in the appended claims, the singular forms include the plural forms. For example, the terms “a,” “an,” and “the” include plural references unless the content clearly dictates otherwise. Additionally, the term “at least” preceding a series of elements is to be understood as referring to every element in the series. The inventions illustratively described herein can suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the future shown and described or any portion thereof, and it is recognized that various modifications are possible within the scope of the invention claimed. Thus, it should be understood that although the present invention has been specifically disclosed by preferred embodiments and optional features, modification and variation of the inventions herein disclosed can be resorted by those skilled in the art, and that such modifications and variations are considered to be within the scope of the inventions disclosed herein. The inventions have been described broadly and generically herein. Each of the narrower species and subgeneric groupings falling within the scope of the generic disclosure also form part of these inventions. This includes the generic description of each invention with a proviso or negative limitation removing any subject matter from the genus, regardless of whether or not the excised materials specifically resided therein. In addition, where features or aspects of an invention are described in terms of the Markush group, those schooled in the art will recognize that the invention is also thereby described in terms of any individual member or subgroup of members of the Markush group. It is also to be understood that the above description is intended to be illustrative and not restrictive. Many embodiments will be apparent to those of in the art upon reviewing the above description. The scope of the invention should therefore, be determined not with reference to the above description, but should instead be determined with reference to the appended claims, along with the full scope of equivalents to which such claims are entitled. Those skilled in the art will recognize, or will be able to ascertain using no more than routine experimentation, many equivalents to the specific embodiments of the invention described. Such equivalents are intended to be encompassed by the following claims.

What is claimed is:

1. A method of treating CTCL comprising applying a combination of administration of an effective amount of hypericin together with a form of visible light photodynamic therapy.

2. The method of claim 1, wherein the effective amount of hypericin is an ointment comprising less than 1% hypericin.

3. The method of claim 1, wherein the form of photodynamic therapy comprises an administration of escalating doses of visible light.

4. The method of claim 3, wherein the escalating doses of visible light starts at about 5 J/cm² and increases to a maximum dose of about 12 J/cm².

5. The method of claim 4, wherein the escalating doses of visible light is increased by about 1 J/cm² from about 1 week to about 3 weeks.

6. The method of claim 4, wherein the escalating doses of visible light will continue until about 12 J/cm² or until light erythema of the lesions is observed.

7. The method of claim 2, wherein the effective amount of hypericin ointment is administered at least once weekly.

8. The method of claim 2, wherein the effective amount of hypericin ointment is administered at least twice weekly.

9. The method of claim 8, wherein the hypericin ointment is applied, then followed by 12-24 hours of absorption into the skin, while covered, and then followed by a light exposure.

10. The method of claim 1, wherein application of the effective amount of hypericin and the form of visible light photodynamic therapy is continued twice weekly for at least 6 weeks.

11. The method of claim 1, wherein the effective amount of hypericin is administered more frequently than the form of visible light photodynamic therapy.

12. The method of claim 1, wherein the effective amount of hypericin is administered less frequently than the form of visible light photodynamic therapy.
